(19) United States
(12) Patent Application Publication
(10) Pub. No.: US 2008/0090967 A1
(43) Pub. Date: Apr. 17, 2008

(54) Modified Alkylresorcinol Resins and Applications Thereof

(75) Inventor: Theodore Harvey Dailey, Kittanning, PA (US)

Correspondence Address: BENJAMIN BAI;JONES DAY/INDSPEC CHEMICAL COMPANY, 717 TEXAS AVENUE, SUITE 3300, HOUSTON, TX, 77002-2712, US

(73) Assignee: INDSPEC Chemical Corporation, Pittsburgh, PA (US)

(21) Appl. No.: 11/870,424

(22) Filed: Oct. 11, 2007

Related U.S. Application Data
(60) Provisional application No. 60/829,394, filed on Oct. 13, 2006.

(51) Int. Cl. C08L 61/00; C07C 39/10

(52) U.S. Cl. 525/139; 568/763

(57) ABSTRACT

A modified alkylresorcinol resin is prepared by reacting one or more alkylresorcinols with one or more aldehydes and optionally an olefinically unsaturated compound. The reaction may be carried out in the presence of a catalyst. The resulted modified alkylresorcinol resin can be used as a methylene acceptor that reacts with methylene donors in vulcanizable rubber compositions.

### PRIOR RELATED APPLICATIONS

This application claims the benefit under 35 U.S.C. §119(e) of U.S. Provisional Patent Application Ser. No. 60/829,394, filed Oct. 13, 2006, which is incorporated herein by reference in its entirety.

### FEDERALLY SPONSORED RESEARCH STATEMENT

Not applicable.

### REFERENCE TO MICROFICHE APPENDIX

Not applicable.

### FIELD OF THE INVENTION

The invention relates to resorcinolic novolak resins obtained by reacting one or more alkylresorcinols with one or more aldehydes and optionally an olefinically unsaturated compound, methods for their synthesis and applications thereof, especially in the formulation of rubber compositions.

### BACKGROUND OF THE INVENTION

In the manufacture of reinforced rubber products, such as automobile tires, it is important to have a good adhesion between the rubber and the reinforcing material. Originally, the adhesion between the rubber and the reinforcing material was promoted by pretreating the reinforcing material with certain adhesives. This was often insufficient and it is common now to incorporate into the rubber during compounding various chemicals that react to improve the adhesion between the rubber and the reinforcing material. This compounding adhesion method is now commonly practiced regardless of whether the reinforcing materials are pretreated with adhesives.

The common compounding adhesion method comprises compounding into the rubber before vulcanization a two part adhesive system. One part is a methylene donor compound. The other part of the adhesive system is a methylene acceptor compound. During the vulcanization step, the methylene donor reacts with the methylene acceptor and the reaction promotes the adhesion between the rubber and the reinforcing material. Furthermore, a proper selection of the methylene donor and methylene acceptor can improve many other properties of the final reinforced rubber products. The methylene donor and the methylene acceptor are compounded into the rubber and thus have a significant effect on the process of making the reinforced rubber products.

Many different methylene acceptors have been tried with various degrees of commercial success. One of the most common methylene acceptors is resorcinol resin. However, the unreacted free resorcinol in the resorcinol resin may present health and environmental problems because the resorcinol may fume under the rubber processing conditions.

Therefore, there is a need to reduce the free resorcinol content in the resorcinol resins.

### SUMMARY OF THE INVENTION

Embodiments of the invention meet at least one of the aforementioned needs in one or more of the following aspects. In one aspect, the invention relates to a modified alkylresorcinol resin prepared by a process comprising reacting a phenolic composition with (a) an olefinically unsaturated compound, and (b) at least an aldehyde, wherein the phenolic composition comprises from about 50 wt. % to about 100 wt. % of one or more alkylresorcinol compounds, from about 0 to about 20 wt. % of resorcinol, and from about 0 to about 10 wt. % of one or more monohydroxyphenol compounds as represented by formula (I)

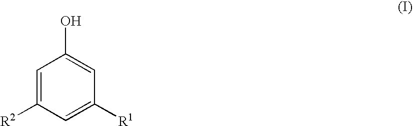

where each of R¹ and R² is independently H, alkyl, or OR³ where R³ is alkyl or aryl.

In some embodiments, the alkylresorcinol compound can be represented by formula (II):

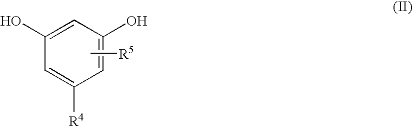

wherein R⁴ is alkyl or substituted alkyl; R⁵ is H, alkyl or substituted alkyl; and R⁵ is in 2, 4 or 6 position of the alkylresorcinol ring. In some embodiments, the phenolic composition is or comprises 5-methylresorcinol, 5-ethylresorcinol, 5-propylresorcinol, 5-butylresorcinol, 5-pentylresorcinol, 5-hexylresoreinol, 5-heptylresorcinol, 5-octylresorcinol, 5-nonylresorcinol, 5-decylresorcinol, 5-undecylresorcinol, 5-dodecylresorcinol, 2-methylresorcinol, 4-methylresorcinol, 2,5-dimethylresorcinol, 4,5-dimethylresorcinol or a combination thereof. In other embodiments, the phenolic composition is or comprises 5-methylresorcinol, 5-ethylresorcinol or a combination thereof. In further embodiments, the phenolic composition comprises from about 1 to about 10 wt. % of resorcinol. In further embodiments, the phenolic composition comprises from about 1 wt. % to about 9 wt. % of the monohydroxyphenol compounds.

In some embodiments, the aldehyde is formaldehyde. In other embodiments, an oxazoidine or other aldehyde substitute is used instead of an aldehyde. In other embodiments, the olefinically unsaturated compound is or comprises styrene, α-methyl styrene, p-methyl styrene, α-chloro styrene, divinyl benzene, vinyl naphthalene, indene, vinyl toluene or a combination thereof. In further embodiments, the olefinically unsaturated compound is styrene.

In another aspect, the invention relates to a resorcinol resin having a structure represented by one of the following formulae:

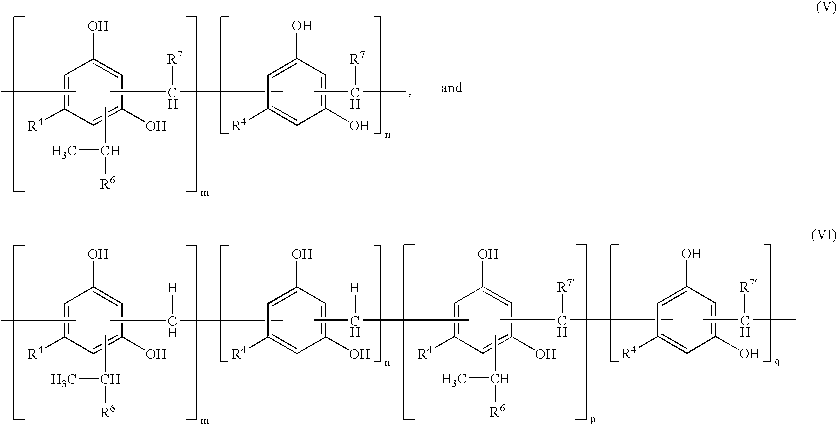

wherein R⁴ is as defined above; R⁶ is alkyl, substituted alkyl, aryl or substituted aryl; R⁷ is H, alkyl, substituted alkyl, aryl or substituted aryl; R^{7′} is alkyl or substituted alkyl; m and n are independently a positive integer; and p and q are independently zero or a positive integer, where the sum of m, n, p, and q is at least 3. It should be noted that the different repeating units illustrated above are randomly distributed in the polymeric backbone. In other words, the modified alkylresorcinol resin is not a block copolymer, but a random copolymer.

In some embodiments, R⁶ is phenyl. In other embodiments, R⁷ is H. In further embodiments, R^{7′} is an alkyl having at least 3 carbon atoms. In particular embodiments, R^{7′} is propyl.

In another aspect, the invention relates to a vulcanizable rubber composition which comprises (I) a rubber component selected from natural rubber, synthetic rubber or combinations thereof, (II) a methylene donor compound, and (III) a methylene acceptor compound comprising at least one of the modified alkylresorcinol resins disclosed herein. In some embodiments, the methylene donor is or comprises hexamethylenetetramine, a methylol melamine, an etherified methylol melamine, an esterified methylol melamine, or a combination thereof. In other embodiments, from about 1 mole % to about 95 mole % of the phenolic groups of the modified alkylresorcinol resin is aralkylated with an olefinically unsaturated compound. In further embodiments, from about 2 mole % to about 90 mole %, from about 3 mole % to about 80 mole %, or from about 4 mole % to about 70 mole % of the phenolic groups of the modified alkylresorcinol resin is aralkylated with an olefinically unsaturated compound. In certain embodiments, from about 30 mole % to about 65 mole % of the phenolic groups of the modified alkylresorcinol resin is aralkylated with an olefinically unsaturated compound.

In another aspect, the invention relates to a process of making a modified alkylresorcinol resin, comprising reacting a phenolic composition with (a) an olefinically unsaturated compound, and (b) at least an aldehyde, wherein the phenolic composition comprises from about 50 wt. % to about 100 wt. % of one or more alkylresorcinol compounds, from about 0 to about 20 wt. % of resorcinol, and from about 0 to about 10 wt. % of one or more monohydroxyphenol compounds as represented by formula (I):

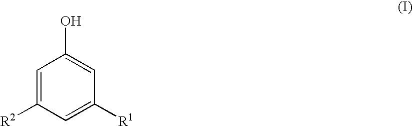

where each of R¹ and R² is independently H, alkyl, or OR³ where R³ is alkyl or aryl. In some embodiments, the molar ratio of the phenolic composition to the olefinically unsaturated compound is from about 1:0.05 to about 1:1.

In other embodiments, the reaction of the phenolic composition with the aldehyde and optionally the olefinically unsaturated compound can occur in the presence of an acid catalyst or a Friedel-Crafts catalyst.

Additional aspects of the invention and characteristics and properties of various embodiments of the invention become apparent with the following description.

### DESCRIPTION OF EMBODIMENTS OF THE INVENTION

In the following description, all numbers disclosed herein are approximate values, regardless whether the word “about” or “approximate” is used in connection therewith. They may vary by 1 percent, 2 percent, 5 percent, or, sometimes, 10 to 20 percent. Whenever a numerical range with a lower limit, RL and an upper limit, RU, is disclosed, any number falling within the range is specifically disclosed. In particular, the following numbers within the range are specifically disclosed: R=RL+k*(RU−RL), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed.

Embodiments of the invention provide a modified alkylresorcinol resin for use in rubber compounding and many other applications. In some embodiments, the modified alkylresorcinol resin disclosed herein comprises a polymeric structure represented by at least one of the following formulae:

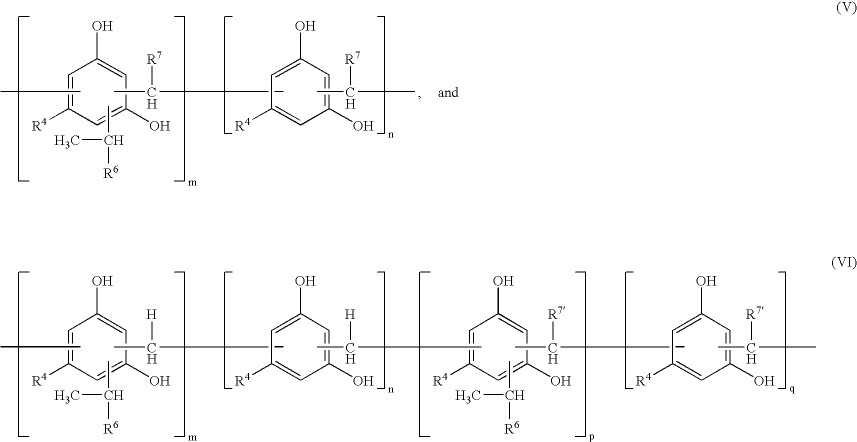

wherein R⁴, R⁶, R⁷ and R^{7′} are as defined above; m and n are independently a positive integer; and p and q are independently zero or a positive integer, where the sum of m, n, p, and q is at least 3. It should be noted that the different repeating units illustrated above are randomly distributed in the polymeric backbone. In other words, the modified alkylresorcinol resin is not a block copolymer, but a random copolymer.

The terminal or end groups of formulae (V) and (VI) may vary between different polymer molecules or units depending on many factors such as the molar ratio of the starting materials, the presence or absence of a chain terminating agent, and the state of the particular polymerization process at the end of the polymerization step. In some embodiments, some of the terminal groups may be H or selected from the group consisting of the following formulae:

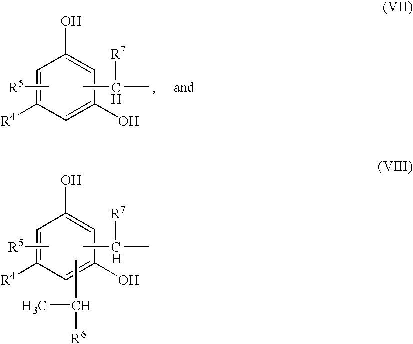

wherein R⁴, R⁵, R⁶ and R⁷ are as defined above. In other embodiments, some of the terminal groups may be selected from the group consisting of the following formulae:

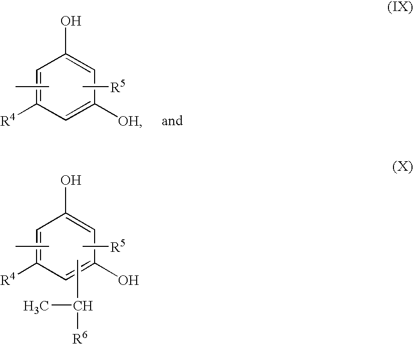

wherein R⁴, R⁵ and R⁶ are as defined above.

The modified alkylresorcinol resin disclosed herein can be prepared or obtained by reacting or contacting a phenolic composition with at least an aldehyde and optionally with at least an olefinically unsaturated compound. In some embodiments, the modified alkylresorcinol resin is prepared by reacting or contacting the phenolic composition with the aldehyde. In other embodiments, the modified alkylresorcinol resin is prepared by reacting or contacting the phenolic composition, the aldehyde and the olefinically unsaturated compound simultaneously or sequentially in any order recognized by a skilled artisan. In further embodiments, the phenolic composition reacts simultaneously with the olefinically unsaturated compound and the aldehyde. In certain embodiments, the phenolic composition reacts sequentially with the olefinically unsaturated compound first and then with the aldehyde. Alternatively, the phenolic composition can react sequentially with the aldehyde first and then with the olefinically unsaturated compound. Furthermore, each of the phenolic composition, the olefinically unsaturated compound and the aldehyde can be independently divided into two or more charges, which can be added to the reaction mixture individually or in any combination.

In some embodiments, the phenolic composition comprises from about 50 wt. % to about 100 wt. %, from about 60 wt. % to about 99 wt. %, from about 65 wt. % to about 95 wt. %, or from about 70 wt. % to about 90 wt. % of one or more alkylresorcinol compounds, based on the total weight of the phenolic composition. In other embodiments, the phenolic composition comprises from about 0 to about 20 wt. %, from about 0.5 wt. % to about 15 wt. %, from about 1 wt. % to about 10 wt. %, or from about 0 to about 5 wt. % of resorcinol, based on the total weight of the phenolic composition. In further embodiments, the phenolic composition comprises from about 0 to about 10 wt. %, from about 1 wt. % to about 9 wt. %, from about 1 wt. % to about 7 wt. %, from about 1 wt. % to about 5 wt. %, or from about 0 to about 3 wt. % of one or more monohydroxyphenol compounds, based on the total weight of the phenolic composition. The monohydroxyphenol compounds can be represented by formula (I)

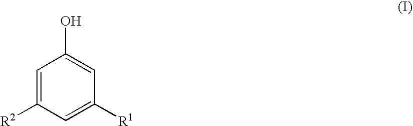

wherein each of R¹ and R² is independently H, alkyl, or OR³ where R³ is alkyl or aryl. In particular embodiments, the phenolic composition comprises from about 50 wt. % to about 100 wt. % of at least one of the alkylresorcinol compounds, from about 0 to about 20 wt. % of resorcinol, and from about 0 to about 10 wt. % of at least one of the monohydroxyphenol compounds. In certain embodiments, the phenolic composition consists essentially of one or more alkylresorcinol compounds disclosed herein.

Any alkylresoreinol compound that reacts with the aldehyde or the olefinically unsaturated compound can be used to prepare the modified alkylresorcinol resin disclosed herein. In some embodiments, the alkylresorcinol compound can be generally represented by formula (II):

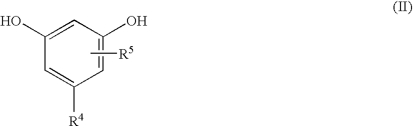

wherein R⁴ is alkyl or substituted alkyl; R⁵ is H, alkyl or substituted alkyl; and R⁵ is in 2, 4 or 6 position of the alkylresorcinol ring. In some embodiments, the alkyl group of the alkylresorcinol compound may be substituted with alkenyl, alkynyl, alkoxy, aryl, aroxyl, halo, —CN, —OH, —NH₂, —CO₂H, —C(═O)NH₂, —C(═O)OCH₃ or the like.

The alkylresorcinol compound of formula (II) can be purchased commercially or prepared by known literature methods. In some embodiments where R⁵ is not H, the alkylresorcinol compound of formula (II) can be prepared by reacting a 5-alkylresorcinol with a carboxylic acid having a formula of R^{5′}—CO₂H where R^{5′} is H, alkyl or substituted alkyl to form the corresponding ketone compound having a keto group —C(═O)—R^{5′} in the presence of zinc chloride catalyst. Next, the keto group can be reduced to —CH₂—R^{5′} (i.e., R⁵) by any suitable reducing agent to form the desired alkylresorcinol compound of formula (II) which can be purified by conventional purification techniques such as extraction, distillation, recrystallization or column chromatography. In other embodiments where R⁵ is H, the 5-alkylresorcinol compound of formula (II) can be purchased commercially or prepared by known literature methods such as those described in Cleaver et al., “Chemical studies off the Proteaceae. IX: Synthesis of 5-alkylresorcinols from aliphatic precursors,” Australian Journal of Chemistry, 29(9), 1989-2001 (1976); and Alonso et al., “Simple synthesis of 5-substituted resorcinols: A revised family of interesting bioactive molecules,” J. Org. Chem., 62, 417 (1997), both of which are incorporated herein by reference.

In some embodiments, R⁵ is H and R⁴ is C₁₋₂₂ alkyl, such as methyl, ethyl, propyl, butyl, pentyl, hexyl, heptyl, octyl, nonyl, decyl, undecyl, dodecyl, tetradecyl, hexadecyl, octadecyl, eicosanyl, docosanyl, and the like. In other embodiments, both R⁵ and R⁴ are independently C₁₋₂₂ alkyl. Some non-limiting examples of suitable alkylresorcinol compounds include 5-methylresorcinol, 5-ethylresorcinol, 5-propylresorcinol, 5-butylresorcinol, 5-pentylresorcinol, 5-hexylresorcinol, 5-heptylresorcinol, 5-octylresorcinol, 5-nonylresorcinol, 5-decylresorcinol, 5-undecylresorcinol, 5-dodecylresorcinol, 2-methylresorcinol, 4-methylresorcinol, 2,5-dimethylresorcinol, 4,5-dimethylresorcinol and combinations thereof. Some non-limiting examples of alkylresorcinol compounds with a substituted R⁴ group include 3,5-dihydroxybenzyl alcohol, methyl 3,5-dihydroxyphenylacetate and combinations thereof.

The amount of the one or more alkylresorcinols in the phenolic composition may vary from about 50 wt. % to about 100 wt. %, from about 50 wt. % to about 90 wt. %, from about 50 wt. % to about 80 wt. %, from about 50 wt. % to about 70 wt. %, from about 60 wt. % to about 100 wt. %, from about 60 wt. % to about 90 wt. %, from about 60 wt. % to about 80 wt. %, from about 70 wt. % to about 100 wt. %, from about 70 wt. % to about 90 wt. %, or from about 80 wt. % to about 100 wt. %, based on the total weight of the phenolic composition. When the phenolic composition comprises two or more alkylresorcinols, each alkylresorcinol can be added to the reaction mixture individually or in combination with other alkylresorcinols.

In some embodiments, the phenolic composition is free of resorcinol. In other embodiments, the phenolic composition is substantially free of resorcinol such that the phenolic composition comprises less than about 0.1 wt. %, about 1 wt. %, about 2 wt. %, about 5 wt. %, about 7.5 wt. %, about 10 wt. %, about 15 wt. % or about 20 wt. % of resorcinol, based on the total weight of the phenolic composition. In further embodiments, the amount of resorcinol in the phenolic composition varies from about 0 to about 20 wt. %, from about 0 to about 15 wt. %, from about 0 to about 10 wt. % or from about 0 to about 8 wt. %, based on the total weight of the phenolic composition.

In some embodiments, the phenolic composition is free of the monohydroxyphenol compounds of formula (I). In other embodiments, the phenolic composition is substantially free of the monohydroxyphenol compounds of formula (I) such that the phenolic composition comprises less than about 0.1 wt. %, about 0.5 wt. %, about 1 wt. %, about 2 wt. %, about 3 wt. %, about 4 wt. %, about 5 wt. %, about 7 wt. % or about 10 wt. % of the monohydroxyphenol compounds of formula (I), based on the total weight of the phenolic composition. In further embodiments, the amount of the monohydroxyphenol compounds of formula (I) in the phenolic composition varies from about 0 to about 10 wt. %, from about 0 to about 5 wt. %, from about 0 to about 3 wt. % or from about 0 to about 2 wt. %, based on the total weight of the phenolic composition.

Any olefinically unsaturated compound that reacts with the alkylresorcinol compounds in the phenolic composition can be used to prepare the modified alkylresorcinol resin disclosed herein. In some embodiments, the olefinically unsaturated compounds include, but are not limited to, vinyl compounds represented by formula (III):

R⁶—CH═CH₂ (III)

wherein R⁶ is alkyl, substituted alkyl such as aralkyl, aryl such as phenyl and naphthyl, or substituted aryl such as alkaryl, alkenaryl, and haloaryl. In some embodiments, the olefinically unsaturated compounds include vinyl aromatic compounds. In other embodiments, the olefinically unsaturated compounds include, but are not limited to, styrene, a-methyl styrene, p-methyl styrene, a-chloro styrene, divinyl benzene, vinyl naphthalene, indene, vinyl toluene, and combinations thereof. In some embodiments, the olefinically unsaturated compound is styrene. When two or more olefinically unsaturated compounds are used, each olefinically unsaturated compound can be added to the reaction mixture individually or in combination with other olefinically unsaturated compounds.

Typically, the molar ratio of the phenolic composition to the olefinically unsaturated compound is between about 1:0.05 to about 1:1. In some embodiments, the molar ratio is from about 1:0,1 to about 1:0.99, from about 1:0.2 to about 1:0.9, from about 1:0.3 to about 1:0.8, from about 1:0.35 to about 1:0.7, from about 1:0.4 to 1:0.65. In other embodiments, the molar ratio is between about 1:0.3 and about 1:0.65.

Any aldehyde that reacts with the alkylresorcinol compounds in the phenolic composition can be used to prepare the modified alkylresorcinol resin disclosed herein. In some embodiments, the aldehyde may be represented by formula (IV):

R⁷—CH═O (IV)

wherein R⁷ is H, alkyl, substituted alkyl such as aralkyl, aryl, or substituted aryl such as alkaryl. The alkyl can be C₁₋₂₂ alkyl such as methyl, ethyl, propyl, isopropyl, butyl, isobutyl, pentyl, isopentyl, hexyl, octyl, nonyl, decyl, dodecyl, tetradecyl, hexadecyl, octadecyl, benzyl and the like. In some embodiments, R⁷ is H, i.e., the aldehyde is formaldehyde. In other embodiments, R⁷ is a C₃₋₂₂ alkyl group. In further embodiments, R⁷ is a C₃ alkyl group. The term “formaldehyde” as used herein also encompasses any substance that can split off or release formaldehyde, such as paraformaldehyde and trioxane.

In some embodiments, the aldehyde is an alkyl aldehyde such as n-butyraldehyde, isobutyraldehyde, valeraldehyde, lauryl aldehyde, palmityl aldehyde, stearyl aldehyde, and combinations thereof. In further embodiments, the aldehyde is formaldehyde, an alkyl aldehyde or a combination thereof. When a mixture of aldehydes are used, they can be added to the reaction mixture individually, simultaneously or sequentially.

In some embodiments, an oxazolidine derivative can be used instead of the aldehyde. In other embodiments, the oxazolidine derivative is an 1-aza-3,7-dioxabicyclo [3.3.0] octane compound represented by formula (XI):

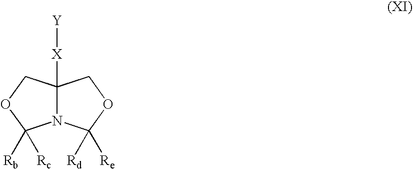

wherein X is a bond, O, S, NRₐ or alkylene; Y is H, alkyl, or OR′ where R′ is H, acyl, alkyl, or aryl; each of Rₐ-Rₑ is independently H, linear or branched alkyl, linear or branched aryl, or cycloalkyl. In some embodiments, the 1-aza-3,7-dioxabicyclo [3.3.0] octane compound is 5-hydroxymethyl-1-aza-3,7-dioxabicyclo [3.3.0] octane or 5-ethyl-1-aza-3,7-dioxabicyclo [3.3.0] octane.

In some embodiments, the oxazolidine derivative is an oxazolidine compound represented by formula (XII):

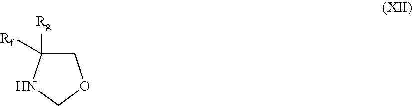

wherein each of R_{f} and R_{g} is independently H, linear or branched alkyl, linear or branched aryl, or cycloalkyl. In some embodiments, the oxazolidine compound is 4,4-dimethyl-1-oxa-3-azacyclopentane.

The condensation reaction between the aldehyde and the phenolic composition can, optionally, be catalyzed. Although the condensation reaction generally can proceed readily without a catalyst when using formaldehyde and other lower molecular weight aldehydes, a catalyst may be desirable when using some of the higher molecular weight aldehydes. Any acidic or basic catalyst known in the art suitable for the condensation reaction of phenolic compounds with aldehydes can be used. Some non-limiting examples of suitable catalysts are disclosed in A. Gardziella, L. A. Pilato, and A. Knop, “Phenolic Resins: Chemistry, Applications, Standardization, Safety and Ecology,” 2^{nd} Edition, Springer-Verlag, New York, Chapter 2, pp. 24-79 (1999), which is incorporated herein by reference.

Generally, the molar ratio of the phenolic composition to the at least an aldehyde can be from about 1:0.2 to about 1:1. In some embodiments, the molar ratio is from about 1:0.3 to about 1:1, from about 1:0.4 to about 1:1, from about 1:0.5 to about 1:1, or from about 1:0.4 to about 1:0.65. In other embodiments, the molar ratio is about 1:0.6, about 1:0.7, about 1:0.8 or about 1:0.9. In some embodiments, the at least an aldehyde comprises formaldehyde and a second aldehyde. The molar ratio of the second aldehyde to formaldehyde can vary from about 0.25:1 to about 3:1. In some embodiments, the molar ratio is from about 0.35:1 to about 2.5:1; from about 0.5:1 to about 2:1; from about 0.6: 1 to about 1.8:1; from about 0.7:1 to about 1.7:1, from about 0.8:1 to about 1.6:1; from about 0.9:1 to about 1.5:1; or from about 1:1 to about 1.2:1.

The modified alkylresorcinol resins disclosed herein may have at least 10 mole percent of the alkylresorcinol groups of the alkylresorcinol compounds aralkylated with one or more olefinically unsaturated compounds, such as styrene, α-methyl styrene, p-methyl styrene, α-chloro styrene, divinyl benzene, vinyl naphthalene, indene, vinyl toluene and combinations thereof. In some embodiments, the modified alkylresorcinol resins may have from about 5 to about 100 mole percent, from about 10 to about 90 mole percent, from about 15 to about 80 mole percent, from about 20 to about 75 mole percent, or from about 30 to about 65 mole percent of the alkylresorcinol groups aralkylated. In other embodiments, from about 25 to about 75 mole percent of the alkylresorcinol groups are aralkylated and that the alkylresorcinol groups are only mono-aralkylated. In other embodiments, some of the alkylresorcinol groups are di-aralkylated. The exact amount of aralkyl groups is dictated by the molar ratio used, which may be altered in order to obtain desired properties of the final product. For example, high amounts of aralkyl groups may lower the softening point of the modified alkylresorcinol resins to an undesirable level. In general, the amount of aralkylation is chosen to give a softening point between about 80° C. and about 150° C., preferably between about 80° C. and about 120° C. The amount of aralkylation can also be chosen to maximize the adhesion between the rubber and the reinforcing material, and other properties such as the reactivity of the modified alkylresorcinol resin with the methylene donor, the reactivity of the modified alkylresorcinol resin to the double bonds in the rubber, the amount of fuming, the amount of blooming and the characteristics of the vulcanized product, i.e., the stiffness.

Aralkyl groups may be formed onto the alkylresorcinol groups of an alkylresorcinol-aldehyde resin by the aralkylation reaction between at least one of the olefinically unsaturated compounds with the alkylresorcinol-aldehyde resin. The alkylresorcinol-aldehyde resin can be prepared by reacting at least one of the alkylresorcinol compounds with at least one of the aldehydes disclosed herein. Alternatively, one or more of the alkylresorcinol compound of formula (II) may be first aralkylated with at least one of the olefinically unsaturated compounds and then, alone or with an additional amount of the alkylresorcinol compounds, reacts with one or more of the aldehydes. In some embodiments, one or more of the alkylresorcinol compounds are first aralkylated with at least one of the olefinically unsaturated compounds and then the aralkylated alkylresorcinol compounds and an additional amount of the alkylresorcinol compounds react with one or more of the aldehydes.

The aralkylation of the alkylresorcinol compounds with the olefinically unsaturated compounds can be carried out in the presence or absence of a solvent. Any suitable solvent that can dissolve both alkylresorcinol compound and the olefinically unsaturated compound can be used. Non-limiting examples of suitable solvents include benzene, toluene, xylene, ethylbenzene and combinations thereof.

Optionally, the aralkylation reaction between the olefinically unsaturated compound and the alkylresorcinol compound can be catalyzed. Some non-limiting examples of suitable catalysts include Friedel-Crafts catalysts, acid catalysts and combinations thereof. Some non-limiting examples of the acid catalysts include inorganic acids (e.g., hydrochloric acid, sulfuric acid, phosphoric acid and phosphorous acid), alkyl sulfonic acids (e.g., methane sulfonic acid), aryl sulfonic acids (e.g., benzene sulfonic acid, benzene disulfonic acid, toluene sulfonic acid and xylene sulfonic acid), and combinations thereof. In some embodiments, the catalyst is an aryl sulfonic acid. In other embodiments, the amount of the catalyst is in the range of about 0.01 parts to about 10 parts of catalyst per 100 parts of the alkylresorcinol compound. The aralkylation reaction is generally carried out at temperatures between about 50° C. to about 180° C.

To prepare the modified alkylresorcinol resin disclosed herein, at least one of the alkylresorcinol compounds are required to react with at least one of the aldehydes. In some embodiments, this condensation reaction can take place with or without the alkylresorcinol compounds being aralkylated. In other embodiments, this condensation reaction can take place before or after the alkylresorcinol compounds are aralkylated. In further embodiments, this condensation reaction takes place after the aralkylation reaction. The condensation reaction may be carried out in the absence or presence of a catalyst. In some embodiments, the condensation reaction takes place in the presence of at least one of the acid catalysts as set forth above. The reaction may preferably be carried out in the range of about 50° C. to about 200° C. The use of a solvent is optional and suitable solvents may be the same as those set forth earlier.

In some embodiments, one or more of the alkylresorcinol compounds and styrene are reacted at a molar ratio of 1 mole of the alkylresorcinol compounds to 0.3 to 0.65 moles of styrene in presence of an acid catalyst at about 120° C. Thereafter, an alkyl aldehyde is added first to the reaction mixture at a molar ratio of 0.2 to 0.45; and then formaldehyde is added at a molar ratio of 0.2 to 0.4. After the reaction mixture reacts at about 100° C. for about 1 hour to about 24 hours, the reaction product is dehydrated.

In other embodiments, one or more of the alkylresorcinol compounds and formaldehyde undergo a condensation reaction at a molar ratio of 1 mole of the alkylresorcinol compounds to 0.5 to 0.7 moles of the total aldehyde (i.e. formaldehyde and alkyl aldehyde) at about 100° C. The condensation reaction product is then dehydrated at atmospheric pressure at about 140° C. Styrene at a molar ratio of 0.30 to 0.65 is then added to aralkylate part of the condensation reaction products at about 140° C.-150° C. Either the condensation reaction or the aralkylation reaction may be run in the presence of a suitable catalyst as set forth above. In some embodiments, the catalysts for the aralkylation reaction and the condensation reaction are the same.

As mentioned above, a vulcanizable rubber composition can be prepared by using the modified alkylresorcinol resin as the methylene acceptor. The vulcanizable rubber composition comprises: (I) a rubber component selected from natural and synthetic rubbers; and (II) a methylene donor compound; and (III) a methylene acceptor comprising the modified alkylresorcinol resin disclosed herein. Optionally, the rubber composition may further comprise (IV) a vulcanizing agent, such as sulfur; and (V) one or more rubber additives.

The rubber component can be a natural rubber, a synthetic rubber or a combination thereof. Non-limiting examples of suitable synthetic rubber polymers include the butadiene polymers such as polybutadiene, isobutylene rubber (butyl rubber), ethylene-propylene rubber (EPDM), neoprene (polychloroprene), polyisoprene, copolymers of 1,3-butadiene or isoprene with monomers such as styrene, acrylonitrile and methyl methacrylate as well as ethylene/propylene/diene monomer (EPDM) and in particular ethylene/propylene/dicyclopentadiene terpolymers. Non-limiting examples of suitable butadiene polymers include those polymers having rubber-like properties, prepared by polymerizing butadiene alone or with one or more other polymerizable ethylenically unsaturated compounds, such as styrene, methylstyrene, methyl isopropenyl ketone and acrylonitrile. The butadiene may be present in the mixture in an amount of at least 40% of the total polymerizable material.

The methylene donor component can be any compound that is capable of reacting with the methylene acceptor used in the rubber compound formulations. Examples of suitable methylene donors include, but are not limited to, hexamethylenetetramine (HEXA or HMT), a methylol melamine, an etherified methylol melamine such as hexamethoxymethylmelamine (HMMM), an esterified methylol melamine, oxazolidine derivatives, N-methyl-1,3,5-dioxazine or a combination thereof. Other suitable methylene donors are described in U.S. Pat. No. 3,751,331, which is incorporated by reference herein in its entirety. The methylene donor is usually present in concentrations of from about 0.5 to about 15 parts per one hundred parts of rubber, preferably from about 0.5 to about 10 parts per one hundred parts of rubber. The weight ratio of methylene donor to methylene acceptor may vary. But, in general, the weight-ratio will range from about 1:10 to about 10:1. Preferably, the weight ratio of methylene donor to methylene acceptor ranges from about 1:3 to about 3:1.

The vulcanizable rubber composition may include a vulcanizing agent, such as sulfur. Examples of suitable sulfur vulcanizing agents include elemental sulfur or sulfur donating vulcanizing agents. Preferably, the sulfur vulcanizing agent is elemental sulfur.

The vulcanizable rubber composition may also include one or more additives such as carbon black, zinc oxide, silica, antioxidants, stearates, accelerators, oils, adhesion promoters, cobalt salts, stearic acid, fillers, plasticizers, waxes, processing oils, retarders, antiozonants and the like. Accelerators can be used to control the time and/or temperature required for the vulcanization and to improve the properties of the vulcanizate. Suitable accelerators include, but are not limited to, amines, disulfides, guanidines, thioureas, thiazoles, thiurams, sulfenamides, dithicarbonates and zanthates. In some embodiments, the primary accelerator is a sulfenamide such as N,N-dicylohexyl-2-benzenethiazole sulfonamide. Any cobalt compound that can promote the adhesion of rubber to metal, such as stainless steel, may be used. Suitable cobalt compounds include, but are not limited to, cobalt salts of fatty acids and other carboxylic acids, such as stearic acid, palmitic, oleic, linoleic, and the like; cobalt salts of aliphatic or alicyclic carboxylic acids having 6 to 30 carbon atoms such as cobalt neodecanoate; cobalt salts of aromatic carboxylic acids such as cobalt naphthenate; cobalt halides such as cobalt chloride; and organo-cobalt-boron complexes such as MANOBOND® 680C from OM Group, Inc., Cleveland, Ohio.

In some embodiments, the vulcanizable rubber composition can be prepared by mixing a rubber material, carbon black, zinc oxide, lubricants and a methylene acceptor in a Banbury mixer at a temperature of about 150° C. The resulting masterbatch is then compounded on a standard 2-roll rubber mill with at least a sulfur accelerator and a methylene donor. Next, the rubber composition can be shaped and cured. Other methods of preparing of rubber compositions and their formulations are described in U.S. Pat. Nos. 6,875,807; 6,605,670; 6,541,551; 6,472,457; 5,945,500; and 5,936,056; all of which are incorporated herein by reference.

The vulcanizable rubber compositions based on the above resins may be used in the preparation of composite products for the manufacture of tires, power belts, conveyor belts, printing rolls, rubber shoe heels and soles, rubber wringers, automobile floor mats, mud flaps for trucks, ball mill liners, and the like. The rubber compound described herein may also be used in the tire applications, for example, as a wire coat or bead coat. Any form of the cobalt compounds known in the art to promote the adhesion of rubber to metal, such as stainless steel, may be used. Suitable cobalt compounds which may be employed include cobalt salts of fatty acids such as stearic acid, palmitic, oleic, linoleic and the like; cobalt salts of aliphatic or alicyclic carboxylic acids having 6 to 30 carbon atoms; cobalt chloride, cobalt naphthenate, cobalt neodecanoate, and an organo-cobalt-boron complex commercially available under the trade name Monobond C.

In some embodiments, the vulcanizable rubber composition further comprises a rubber reinforcing material. Any rubber reinforcing material that can strengthen rubber materials can be used, including, but not limited to, polyesters, polyamides (e.g., nylons and aramid), polyvinyl alcohol, carbon, glass, steel (brass, zinc or bronze plated), polybenzoxazole, rayon, and other organic or inorganic compositions. These rubber reinforcing materials may be in the form of filaments, fibers, cords, or fabrics. In some embodiments, the rubber reinforcing material can be a steel cord coated by brass, zinc, bronze or a combination thereof.

While not necessary, the rubber reinforcing material can be coated with an adhesive composition before it is combined with an uncured rubber composition. Any adhesive composition that can enhance the adhesion between the reinforcing material and the cured rubber component can be used. For examples, certain suitable adhesive compositions for enhancing the adhesion between rubber materials and rubber reinforcing materials are disclosed in U.S. Pat. Nos. 6,416,869; 6,261,638; 5,789,080; 5,126,501; 4,588,645; 4,441,946; 4,236,564; 4,051,281; 4,052,524; and 4,333,787, which are incorporated herein by reference in their entirety. These adhesive compositions can be used according to the methods taught therein, with or without modifications.

Fabricated articles can be made from the vulcanizable rubber composition disclosed herein. Non-limiting examples of the fabricated article include tires, belts such as power transmission belts, conveyor belts and V-belts, hoses such as pneumatic and hydraulic hoses, printing rolls, rubber shoe heels, rubber shoe soles, automobile floor mats, truck mud flaps and ball mill liners.

In some embodiments, the fabricated rubber article can be prepared according to the following method which comprises the steps of (1) obtaining a vulcanizable rubber composition as described above mixed with a cross-linking agent; (2) embedding in the vulcanizable rubber composition a rubber reinforcing material; and (3) effecting cross-linking of the rubber composition.

The following examples are presented to exemplify embodiments of the invention. All numerical values are approximate. When numerical ranges are given, it should be understood that embodiments outside the stated ranges may still fall within the scope of the invention. Specific details described in each example should not be construed as necessary features of the invention.

### EXAMPLES

In the following examples, various methylene acceptor resins were prepared and evaluated in a black natural rubber compound to assess and compare their performance against PENACOLITE® Resin B-19-S and/or PENACOLITE® Resin B-20-S, both of which are available from INDSPEC Chemical Corporation, Pittsburgh, Pa., for steel-wire adhesion and cured rubber compound dynamic properties. Vulcanizable rubber compositions, having the general formulation shown in Table 1, were prepared in a 3-stage mixing procedure. These vulcanizable rubber compositions were then used to evaluate the adhesion and reinforcing effects of the modified alkylresorcinol resins as methylene acceptors in combination with the methylene donor hexamethoxymethylmelamine (HMMM). The methylene acceptor/donor ratio was kept at 3:2 by weight with a combined loading of 5 parts by weight in the vulcanizable rubber compositions.

TABLE 1
General Formulation of Vulcanizable Rubber
Compositions Used in Testing

Parts
(by wt.)

First Stage

| | | |
|---|---|---|
| 1 | Natural Rubber | 100 |
| 2 | Carbon Black | 55 |
| 3 | Zinc Oxide | 8 |
| 4 | Stearic Acid | 1 |
| 5 | N-(1,2-Dimethylbutyl)-N′-phenyl-p-phenylenediamine | 2 |
| 6 | 1,2-Dihydro-2,2,4-trimethylquinoline | 1 |
| 7 | Pre-Vulcanization Inhibitor (N-(Cyclohexylthio)phthalimide | 0.2 |

Second Stage

| | | |
|---|---|---|
| 8 | Methylene Acceptor (Resin) | 3 |
| 9 | Cobalt Salt (MANOBOND ® 680C, 22% Co) | 0.44 |

Third Stage

| | | |
|---|---|---|
| 10 | Insoluble Sulfur (80%) | 5 |
| 11 | N,N-Dicyclohexyl-2-benzenethiazole sulfenamide | 1 |
| 12 | Methylene Donor (HMMM, 72% Active) | 2.78 |

In the first stage, a rubber masterbatch was prepared by mixing the ingredients listed under the first stage in Table 1 at about 150° C. in a Banbury mixer. In the second stage, a cobalt salt MANOBOND® 680 C and a methylene acceptor, such as PENACOLITE® Resin B-19-S, PENACOLITE® Resin B-20-S and the modified alkylresorcinol resins disclosed herein, were mixed with an appropriate amount of the masterbatch on a two-roll mill at about 121° C. In the third stage, appropriate amounts of the insoluble sulfur, accelerator and HMMM as indicated in Table 1 were added to the two-roll mill and the mixture was mixed at 95° C. The vulcanizable rubber compositions were conditioned overnight in a constant temperature room at about 23° C. and about 50% relative humidity. The vulcanizable rubber compositions were then tested for Mooney viscosity, rheometer cure, wire adhesion, dynamic mechanical properties, Shore A hardness values, tensile properties and Die C Tear properties.

The Mooney viscosities were measured using an Alpha Technologies MV2000 Mooney Viscometer according to ASTM D1 646-04, which is incorporated herein by reference. Mooney viscosity is defined as the shearing torque resisting rotation of a cylindrical metal disk (or rotor) embedded in rubber within a cylindrical cavity. The cure properties were measured with an Alpha Technologies MDR2000 Rheometer at 150° C., 0.5° arc and 1.67 Hz according to ASTM D 5289, which is incorporated herein by reference. The samples were cured at 100° C. and 150° C., respectively for the Mooney viscosity and cure property measurements.

Wire adhesion properties were determined according to ASTM D 2229-02 using brass plated steel cord (Wire: Bekaert 3×0.2+6×0.35 with 64% copper plating) embedded 19 mm into the rubber pad. The samples were cured to the Rheometer t′100 plus seven minutes at 150° C. and then tested under unaged condition, steam-aged condition and humidity-aged condition. ASTM D 2229-02 is incorporated herein by reference

Dynamic mechanical properties, such as Dynamic stiffness G′ and tangent delta, were measured using a TA Instruments ARES-RDA at both 23° C. and 60° C. The tests were run at a frequency of 1.0 Hz at 2% torsional shear strain. A rectangular specimen 18 mm long, 12 mm wide and 4 mm thick was used.

The Shore A hardness values were measured according to ASTM-D2240-03, which is incorporated herein by reference. The tensile properties were measured according to ASTM D412, which is incorporated herein by reference. The Die C Tear properties were measured according to ASTM D624C, which is incorporated herein by reference.

The softening points of the modified alkylresorcinol resins were measured according to the following method with reference to the latest edition of ASTM E 28 and ASTM D 3104, which are incorporated by reference herein in their entirety.

Apparatus: cups pitch type drilled to 0.257″ Opening (F drill); a 440 stainless steel ball (0.2500″ in diameter and must pass through cups); a Mettler softening point apparatus comprising (1) a control unit Model FP-90 or equivalent, (2) a furnace Model FP-83 or equivalent, and (3) cartridge assemblies; a timer; porcelain evaporating dishes (about 3″ in diameter); and a hot plate. For calibration of the Mettler apparatus, see ASTM D 3104, which is incorporated by reference herein.

Procedures: melt 15 grams of resin in a porcelain or aluminum evaporating dish. At 600-650° F., surface temperature of hot plate, melting time is approximately 4 minutes. Overheating should be avoided. When the resin is melted, pour into cups that have been preheated to at least the temperature of the molten resin. The quantity of resin poured into the cups should be such that after solidification the excess can be removed with a heated spatula or putty knife. An aluminum plate with holes drilled in it to form a support on the sides and bottom of the cup can be used, or they can be held with forceps when removing excess resin. After the samples have been cooled to room temperature in a desiccator, assemble the cartridge so that the ball rests on the top of the resin. Place the assembled cartridge in the furnace, which has been preset to 85° C. or 10-15° C. below the expected soft point. Set the heating rate at 1° C./min. Turn the cartridge until it locks into position, and wait 30 seconds. Then, initiate operation of softening point apparatus. Read the completed softening point on the indicator. Duplicate determinations should not differ by more than 1.0° C.

The amounts of free resorcinol and alkylresorcinols in the modified alkylresorcinol resins disclosed herein can be measured by any suitable methods, such as LC, GC and HPLC techniques, known in the art. For example, the amounts of free resorcinol and alkylresorcinols in the modified alkylresorcinol resins I-15 listed in Tables 2 and 3 can be and were measured according to the following HPLC procedure. The free resorcinol and alkylresorcinols contents were determined by a reverse phase liquid chromatographic separation using a HPLC system comprising a UV detector at 254 nm, a 10 micro liter fixed loop injection, and a 250 mm×4.6 mm Phenomenex Prodigy ODS(2) column or equivalent. The HPLC system was programmed for a 35 minute gradient elution of the mobile phase at a flow rate of 1 ml/minute, a temperature of 30° C., and an injection volume of 10 micro liters. The mobile phase was a mixture of HPLC grade water (W) and HPLC grade acetonitrile (A), the proportion of which was linearly programmed to range from 80-85% W and 15-20% A at the start to 15-20% W and 80-85% A, then back to the original composition of 80-85% W and 15-20% A at the finish. An external standard calibration method was used where standards were prepared for calibration by weighing known amounts of resorcinol and alkylresorcinols. These known concentrations were injected into the HPLC system to determine retention times and to calculate response factors using techniques known to those skilled in the art. Samples were weighed, dissolved and then diluted in 95% ethanol. Each sample was injected and the peak areas were transformed by calculation to weight percent concentrations of the corresponding components using the response factors generated in calibration.

### Example 1

### Synthesis of Alkylresorcinol-Formaldehyde and Alkylresorcinol-Resorcinol-Formaldehyde Resins

TABLE 2
Synthesis of Alkylresorcinol-Formaldehyde Resins.

| | Resin Number | | | | | | | |
|---|---|---|---|---|---|---|---|---|
| | 1 | 2 | 3 | 4 | 5 | 6 | 7 | 8 |
| Raw Materials (Mole) | | | | | | | | |
| Resorcinol | 0 | 0 | 0 | 0.18 | 0.36 | 0.54 | 1.20 | 0 |
| Resorcinol Homopolymer | 0 | 0 | 0 | 0 | 0 | 0 | 0 | 0.75 |
| HONEYOL | 1.67 | 1.67 | 1.67 | 1.50 | 1.30 | 1.17 | 0.67 | 0.88 |
| Formaldehyde | 0.88 | 0.79 | 0.84 | 0.84 | 0.85 | 0.86 | 0.83 | 1.01 |
| Resin Properties | | | | | | | | |
| Softening Point (° C.) | 112.9 | 96.3 | 103.3 | 103.1 | 100.7 | 98.7 | 83.6 | 115.5 |
| Free Resorcinol (wt. %) | 0.6 | 1.1 | 0.5 | 2.7 | 5.0 | 7.5 | 17.0 | 5.6 |
| Free 2-methylresorcinol (wt. %) | <0.1 | <0.1 | 0.51 | 0.38 | 0.53 | 0.49 | <0.05 | <0.05 |
| Free 5-methylresorcinol (wt. %) | 5.6 | 7.6 | 7.2 | 6.9 | 6.6 | 5.7 | 3.9 | 1.9 |
| Free 2,5-dimethylresorcinol (wt. %) | 1.6 | 1.7 | 1.9 | 1.6 | 1.5 | 1.2 | 0.9 | 0.5 |

Note:
HONEYOL was obtained from VKG Oil AS, Kohtla-Jarve, Estonia.

Alkylresorcinol-formaldehyde resins 1-3 and alkylresorcinol-resorcinol-formaldehyde resins 4-8 were prepared according to the general procedure as described below. The molar charges of the ingredients for each resin are listed in Table 2 above.

First, HONEYOL, a mixture of HONEYOL and resorcinol or a mixture of HONEYOL, resorcinol and resorcinol homopolymer was added to a reaction flask fitted with a stirrer, heating mantle and a condenser. The reflux from the condenser was set to return to the reaction flask. The resorcinol component was heated until molten and stirred. Once the temperature of resorcinol component was at or above 100° C., formaldehyde was added drop-wise over 1 to 3 hours, so as not to exceed the capacity of the condenser. The reflux from the condenser was returned to the flask to cool the reaction mixture.

After the specified amount of formaldehyde was added, the condenser output was re-routed to a distillate receiver and temperature was increased to distill water of reaction from the resin. Distillation continued at atmospheric pressure until reaction mass temperature reached 140° C. to 145° C., then vacuum was applied to the flask to remove the remaining water. The batch was vacuum distilled to about 685 torr of vacuum and a temperature of 155° C. to 165° C., or until water content was below 2 wt. %.

After the vacuum was released, the resin was discharged and cast in a thin layer on a tray. After hardened, the resin was stored in a sealed jar. After the resin was tested for softening points and the amounts of free resorcinol and alkylresorcinols such as 2-methylresorcinol, 5-methylresorcinol and 2,5-dimethylresoreinol, the resin was used in the rubber compounding experiments discussed below. The softening points and the amounts of free resorcinol, 2-methylresorcinol, 5-methylresorcinol and 2,5-dimethylresorcinol of alkylresorcinol-formaldehyde resins 1-3 and alkylresorcinol-resorcinol-formaldehyde resins 4-8 are listed in Table 2 above.

### Example 2

### Synthesis of Alkylresorcinol-Styrene-Formaldehyde Resins

TABLE 3
Synthesis of Alkylresorcinol-Styrene-Formaldehyde Resins

| | Resin Number | | | | | |
|---|---|---|---|---|---|---|
| | 9 | 10 | 11 | 12 | 13 | 14 |
| Raw Materials (Mole) | | | | | | |
| HONEYOL | 1.49 | 1.49 | 1.49 | 1.49 | 1.49 | 1.49 |
| Styrene | 0.80 | 0.64 | 0.64 | 0.38 | 0.25 | 0.50 |
| Formaldehyde | 0.73 | 0.73 | 0.73 | 0.73 | 0.73 | 0.73 |
| Resin Properties | | | | | | |
| Softening Point (° C.) | 95.4 | 100.7 | 98.0 | 97.8 | 99.1 | 98.7 |
| Free resorcinol (wt. %) | 0.04 | 0.14 | 0.05 | 0.17 | 0.14 | 0.03 |
| Free 2-methylresorcinol | <0.05 | <0.05 | <0.01 | <0.01 | <0.01 | <0.01 |
| (wt. %) | | | | | | |
| Free 5-methylresorcinol | 0.6 | 1.4 | 1.2 | 2.9 | 3.9 | 1.7 |
| (wt. %) | | | | | | |
| Free 2,5-dimethylresorcinol | 0.4 | 0.5 | 0.7 | 1.3 | 1.5 | 1.0 |
| (wt. %) | | | | | | |

Note:
HONEYOL was obtained from VKG Oil AS, Kohtla-Jarve, Estonia.

Alkylresorcinol-styrene-formaldehyde resins 9-14 were prepared according to the general procedure as described below. The molar charges of the ingredients for each resin are listed in Table 3 above.

First, about 84% of HONEYOL and p-toluene sulfonic acid at a level equal to 0.2 wt. % of HONEYOL were added to a reaction flask fitted with a stirrer, heating mantle and a condenser. The reflux from the condenser was set to return to the reaction flask. The HONEYOL was heated to 125° C. and stirred. Once the temperature of HONEYOL reached 125° C., styrene was added drop-wise to the flask over about 1 to 2 hours, while taking care to maintain temperature between 125° C. and 135° C.

Once the styrene had been added, the reaction mixture was briefly heated to 150° C. to assure the reaction was complete. The remainder of the HONEYOL charge was added. The p-toluene sulfonic acid was neutralized with an equal molar amount of sodium hydroxide solution and then cooled to about 100° C.

Next, formaldehyde was added drop-wise over 1 to 3 hours, so as not to exceed the capacity of the condenser. The reflux from the condenser was returned to the flask to cool the reaction material.

After all formaldehyde was added, the condenser output was re-routed to a distillate receiver and temperature was increased to distill water of reaction from the resin. Distillation continued at atmospheric pressure until reaction mass temperature reached 140° C. to 145° C., then vacuum was applied to the flask to remove the remaining water. The batch was vacuum distilled to about 685 torr of vacuum and a temperature of 155° C. to 165° C., or until water content was below 2 wt. %.

After the vacuum was released, the resin was discharged and cast in a thin layer on a tray. After hardened, the resin was stored in a sealed jar. After the resin was tested for softening points and the amounts of free resorcinol and alkylresorcinols such as 2-methylresorcinol, 5-methylresorcinol and 2,5-dimethylresorcinol, the resin was used in the rubber compounding experiments discussed below. The softening points and the amounts of free resorcinol, 2-methylresorcinol, 5-methylresorcinol and 2,5-dimethylresorcinol of alkylresorcinol-styrene-formaldehyde resins 9-14 are listed in Table 2 above.

### Example 3

### Testing of Alkylresorcinol-Formaldehyde Resins

Alkylresorcinol-formaldehyde resins 1 and 2 were used to prepare rubber compounds B and C respectively according to the procedures described above and the acceptor/donor ratios as shown in Table 4 below. Rubber compounds A, D and E were also prepared similarly as comparisons using PENACOLITE® Resin B-19-S, VKG SF-281 and VKG AFES respectively as the methylene acceptor. The physical properties of the rubber compounds were evaluated accordingly and the testing results are listed in Table 4 below. The data in Table 4 show that the Mooney viscosity, rheometer cure, wire adhesion, dynamic mechanical properties, Shore A hardness values, tensile properties and Die C Tear properties of compounds A-E are comparable

TABLE 4

| | Compound | | | | |
|---|---|---|---|---|---|
| | A | B | C | D | E |
| Methylene Acceptor | PENACOLITE ® | Resin 1 | Resin 2 | VKG | VKG |
| | Resin B-19-S | | | SF-281 | AFES |
| Methylene Donor | HMMM | HMMM | HMMM | HMMM | HMMM |
| Weight Ratio; Acceptor/Donor, phr | 3/2 | 3/2 | 3/2 | 3/2 | 3/2 |
| Mooney Viscosity at 100° C. | | | | | |
| ML (1 + 4) | 60.1 | 61.0 | 58.5 | 58.9 | 59.0 |
| Rheometer Cure at 150° C. | | | | | |
| M_{H}, dN-m | 36.40 | 40.98 | 42.86 | 38.21 | 36.68 |
| M_{L}, dN-m | 2.79 | 3.06 | 2.91 | 2.84 | 2.79 |
| tₛ2, minutes | 2.01 | 1.65 | 1.67 | 2.10 | 2.37 |
| t′90, minutes | 17.13 | 16.77 | 16.00 | 17.62 | 18.81 |
| Wire Adhesion, N (% Rubber Coverage) | | | | | |
| Unaged | 1181(95) | 1225(90) | 1242(85) | 1139(85) | 1086(80) |
| Steam, 24 Hours @ 120° C. | 1267(100) | 1244(100) | 1121(90) | 1129(90) | 1037(80) |
| Humidity, 21 Days, 85° C./95% RH | 1130(95) | 1108(95) | 1129(95) | 1150(90) | 1072(80) |
| Dynamic Mechanical Properties | | | | | |
| G′ at 2% strain, MPa, @ 23° C. | 11.65 | 14.65 | 14.90 | 14.34 | 14.35 |
| Tan Delta at 2% strain | 0.178 | 0.169 | 0.161 | 0.167 | 0.170 |
| G′ at 2% strain, MPa, @ 60° C. | 10.05 | 12.92 | 13.12 | 12.43 | 12.42 |
| Tan Delta at 2% strain | 0.165 | 0.159 | 0.155 | 0.158 | 0.167 |
| Shore A Hardness | 81 | 84 | 84 | 84 | 83 |
| Tensile Properties | | | | | |
| 100% Modulus, MPa | 4.64 | 5.19 | 5.18 | 5.11 | 5.28 |
| Tensile Strength, MPa | 25.6 | 26.4 | 25.7 | 26.2 | 25.7 |
| Elongation, % | 448 | 457 | 446 | 469 | 458 |
| Die C Tear, N/mm | 111.3 | 112.3 | 112.7 | 116.4 | 113.3 |

Note:
PENACOLITE ® Resin B-19-S was obtained from INDSPEC Chemical Corporation, Pittsburgh, PA. VKG SF-281 and VKG AFES were obtained from VKG Oil AS, Kohtla-Jarve, Estonia.

### Example 4

### Testing of Alkylresorcinol-Formaldehyde Resins and Alkylresorcinol-Resorcinol-Formaldehyde Resins

Alkylresorcinol-formaldehyde resin 3 and alkylresorcinol-resorcinol-formaldehyde resins 4-7 were used to prepare rubber compounds G, H, I, J and K respectively according to the procedures described above and the acceptor/donor ratios as shown in Table 5 below. Rubber compound F was also prepared similarly as a comparison using PENACOLITE® Resin B-19-S as the methylene acceptor. The physical properties of the rubber compounds were evaluated accordingly and the testing results are listed in Table 5 below. The data in Table 5 show that the Mooney viscosity, rheometer cure, wire adhesion, dynamic mechanical properties, Shore A hardness values, tensile properties and Die C Tear properties of compounds F-K are comparable.

TABLE 5

| | Compound | | | | | |
|---|---|---|---|---|---|---|
| | F | G | H | I | J | K |
| Methylene Acceptor | B-19-S* | Resin 3 | Resin 4 | Resin 5 | Resin 6 | Resin 7 |
| Methylene Donor | HMMM | HMMM | HMMM | HMMM | HMMM | HMMM |
| Weight Ratio; Acceptor/Donor, phr | 3/2 | 3/2 | 3/2 | 3/2 | 3/2 | 3/2 |
| Mooney Viscosity at 100° C. | | | | | | |
| ML (1 + 4) | 58.6 | 58.8 | 58.1 | 57.8 | 57.7 | 57.1 |
| Rheometer Cure at 150° C. | | | | | | |
| M_{H}, dN-m | 37.17 | 42.39 | 42.26 | 42.55 | 42.36 | 42.21 |
| M_{L}, dN-m | 2.59 | 2.81 | 2.74 | 2.74 | 2.68 | 2.54 |
| tₛ2, minutes | 2.12 | 1.72 | 1.69 | 1.77 | 1.78 | 1.99 |
| t′90, minutes | 17.96 | 16.79 | 16.66 | 16.79 | 16.78 | 17.25 |
| Wire Adhesion, N (% Rubber Coverage) | | | | | | |
| Unaged | 1198(90) | 1250(95) | 1237(95) | 1193(90) | 1140(95) | 1178(95) |
| Steam, 24 Hours @ 120° C. | 1268(95) | 1147(80) | 1155(85) | 1267(100) | 1233(100) | 1220(95) |
| Humidity, 21 Days, 85° C./95% RH | 1252(100) | 1303(100) | 1278(100) | 1280(100) | 1240(100) | 1258(100) |
| Dynamic Mechanical Properties | | | | | | |
| G′ at 2% strain, MPa @ 23° C. | 12.91 | 15.51 | 15.18 | 15.16 | 15.09 | 14.73 |
| Tan Delta at 2% strain | 0.186 | 0.168 | 0.173 | 0.177 | 0.176 | 0.183 |
| G′ at 2% strain, MPa @ 60° C. | 11.07 | 13.14 | 13.63 | 13.45 | 13.25 | 12.64 |
| Tan Delta at 2% strain | 0.176 | 0.16 | 0.166 | 0.165 | 0.165 | 0.173 |
| Shore A Hardness | 82 | 84 | 84 | 84 | 83 | 84 |
| Tensile Properties | | | | | | |
| 100% Modulus, MPa | 4.32 | 4.95 | 4.85 | 4.76 | 4.70 | 4.64 |
| Tensile Strength, MPa | 25.8 | 27.3 | 27.0 | 27.2 | 26.8 | 27.2 |
| Elongation, % | 469 | 476 | 470 | 483 | 478 | 485 |
| Die C Tear, N/mm | 119.4 | 125.0 | 114.1 | 115.8 | 120.5 | 116.6 |

Note:
*B-19-S is PENACOLITE ® Resin B-19-S obtained from INDSPEC Chemical Corporation, Pittsburgh, PA.

### Example 5

### Testing of Alkylresorcinol-Resorcinol-Formaldehyde Resins

Alkylresorcinol-resorcinol-formaldehyde resins 7 and 8 were used to prepare rubber compounds M and N respectively according to the procedures described above and the acceptor/donor ratios as shown in Table 6 below. Rubber compound L was also prepared similarly as a comparison using PENACOLITE® Resin B-19-S as the methylene acceptor. The physical properties of the rubber compounds were evaluated accordingly and the testing results are listed in Table 6 below. The data in Table 6 show that the Mooney viscosity, rheometer cure, wire adhesion, dynamic mechanical properties, Shore A hardness values, tensile properties and Die C Tear properties of compounds L-N are comparable.

TABLE 6

| | Compound | | |
|---|---|---|---|
| | L | M | N |
| Methylene Acceptor | B-19-S* | Resin 7 | Resin 8 |
| Methylene Donor | HMMM | HMMM | HMMM |
| Weight Ratio; Acceptor/Donor, | 3/2 | 3/2 | 3/2 |
| phr | | | |
| Mooney Viscosity at 100° C. | | | |
| ML (1 + 4) | 57.8 | 55.7 | 56.8 |
| Rheometer Cure at 150° C. | | | |
| M_{H}, dN-m | 35.84 | 40.52 | 36.35 |
| M_{L}, dN-m | 2.66 | 2.63 | 2.73 |
| tₛ2, minutes | 1.83 | 1.79 | 1.77 |
| t′90, minutes | 16.61 | 16.23 | 16.81 |
| Wire Adhesion, N | | | |
| (% Rubber Coverage) | | | |
| Unaged | 1267(100) | 1203(100) | 1277(100) |
| Steam, 24 Hours @ 120° C. | 1172(95) | 1177(90) | 1219(95) |
| Humidity, 21 Days, 85° C./95% | 1136(95) | 1196(100) | 1211(95) |
| RH | | | |
| Dynamic Mechanical Properties | | | |
| G′ at 2% strain, MPa @ 23° C. | 11.99 | 11.19 | 13.19 |
| Tan Delta at 2% strain | 0.188 | 0.171 | 0.190 |
| G′ at 2% strain, MPa @ 60° C. | 11.00 | 11.69 | 11.33 |
| Tan Delta at 2% strain | 0.175 | 0.173 | 0.177 |
| Shore A Hardness | 81 | 81 | 80 |
| Tensile Properties | | | |
| 100% Modulus, MPa | 4.62 | 4.81 | 4.76 |
| Tensile Strength, MPa | 26.4 | 25.2 | 26.6 |
| Elongation, % | 450 | 437 | 454 |
| Die C Tear, N/mm | 102.2 | 117.4 | 120.8 |

Note:
*B-19-S is PENACOLITE ® Resin B-19-S obtained from INDSPEC Chemical Corporation, Pittsburgh, PA.

### Example 6

### Testing of Alkylresorcinol-Styrene-Formaldehyde Resins

Alkylresorcinol-styrene-formaldehyde resins 9 and 10 were used to prepare rubber compounds P and Q respectively according to the procedures described above and the acceptor/donor ratios as shown in Table 7 below. Rubber compound O was also prepared similarly as a comparison using PENACOLITE® Resin B-20-S as the methylene acceptor. The physical properties of the rubber compounds were evaluated accordingly and the testing results are listed in Table 7 below. The data in Table 7 show that the Mooney viscosity, rheometer cure, wire adhesion, dynamic mechanical properties, Shore A hardness values, tensile properties and Die C Tear properties of compounds O-Q are comparable.

TABLE 7

| | Compound | | |
|---|---|---|---|
| | O | P | Q |
| Methylene Acceptor | B-20-S* | Resin 9 | Resin 10 |
| Styrene/Alkylresorcinol mole ratio | 0.66:1 | 0.535:1 | 0.43:1 |
| Methylene Donor | HMMM | HMMM | HMMM |
| Weight Ratio; Acceptor/Donor, phr | 3/2 | 3/2 | 3/2 |
| Mooney Viscosity at 100° C. | | | |
| ML (1 + 4) | 56.2 | 56.1 | 57.2 |
| Rheometer Cure at 150° C. | | | |
| M_{H}, dN-m | 34.42 | 33.96 | 35.40 |
| M_{L}, dN-m | 2.57 | 2.63 | 2.74 |
| tₛ2, minutes | 2.58 | 3.04 | 2.37 |
| t′90, minutes | 20.27 | 22.21 | 20.30 |
| Wire Adhesion, N | | | |
| (% Rubber Coverage) | | | |
| Unaged | 1240(95) | 1222(90) | 1237(100) |
| Steam, 24 Hours @ 120° C. | 1215(90) | 1212(95) | 1187(90) |
| Humidity, 21 Days, 85° C./95% | 1199(80) | 1090(80) | 1167(85) |
| RH | | | |
| Dynamic Mechanical Properties | | | |
| G′ at 2% strain, MPa, @ 23° C. | 13.22 | 13.66 | 13.89 |
| Tan Delta at 2% strain | 0.183 | 0.183 | 0.177 |
| G′ at 2% strain, MPa, @ 60° C. | 12.38 | 12.22 | 12.29 |
| Tan Delta at 2% strain | 0.177 | 0.176 | 0.169 |
| Shore A Hardness | 82 | 82 | 83 |
| Tensile Properties | | | |
| 100% Modulus, MPa | 4.84 | 5.14 | 5.06 |
| Tensile Strength, MPa | 26.2 | 25.7 | 26.8 |
| Elongation, % | 438 | 430 | 448 |
| Die C Tear, N/mm | 110.9 | 95.2 | 114.0 |

Note:
*B-20-S is PENACOLITE ® Resin B-20-S obtained from INDSPEC Chemical Corporation, Pittsburgh, PA.

### Example 7

### Testing of Alkylresorcinol-Styrene-Formaldehyde Resins

Alkylresorcinol-styrene-formaldehyde resins 11, 14, 12 and 13 were used to prepare rubber compounds S, T, U and V respectively according to the procedures described above and the acceptor/donor ratios as shown in Table 8 below. Rubber compound R was also prepared similarly as a comparison using PENACOLITE® Resin B-20-S as the methylene acceptor. The physical properties of the rubber compounds were evaluated accordingly and the testing results are listed in Table 8 below. The data in Table 8 show that the Mooney viscosity, rheometer cure, wire adhesion, dynamic mechanical properties, Shore A hardness values, tensile properties and Die C Tear properties of compounds R-V are comparable

TABLE 8

| | Compound | | | | |
|---|---|---|---|---|---|
| | R | S | T | U | V |
| Methylene Acceptor | B-20-S* | Resin 11 | Resin 14 | Resin 12 | Resin 13 |
| Styrene/Alkylresorcinol mole ratio | 0.66:1 | 0.51:1 | 0.4:1 | 0.3:1 | 0.2:1 |
| Methylene Donor | HMMM | HMMM | HMMM | HMMM | HMMM |
| Weight Ratio; Acceptor/Donor, phr | 3/2 | 3/2 | 3/2 | 3/2 | 3/2 |
| Mooney Viscosity at 100° C. | | | | | |
| ML (1 + 4) | 54 | 55 | 55 | 56 | 56 |
| Rheometer Cure at 150° C. | | | | | |
| M_{H}, dN-m | 34.54 | 34.71 | 36.04 | 37.01 | 36.63 |
| M_{L}, dN-m | 2.44 | 2.55 | 2.60 | 2.66 | 2.65 |
| tₛ2, minutes | 2.76 | 2.68 | 2.44 | 2.23 | 2.13 |
| t′90, minutes | 20.69 | 21.64 | 20.57 | 19.55 | 19.13 |
| Wire Adhesion, N (% Rubber Coverage) | | | | | |
| Unaged | 1280(95) | 1181(90) | 1225(90) | 1172(90) | 1163(90) |
| Steam, 24 Hours @ 120° C. | 1375(95) | 1234(95) | 1291(90) | 1172(90) | 1212(90) |
| Humidity, 21 Days, 85° C./95% RH | 1179(95) | 1274(90) | 1274(95) | 1285(95) | 1261(95) |
| Dynamic Mechanical Properties | | | | | |
| G′ at 2% strain, MPa, @ 23° C. | 16.5 | 15.95 | 16.58 | 16.67 | 15.36 |
| Tan Delta at 2% strain | 0.185 | 0.181 | 0.18 | 0.177 | 0.175 |
| G′ at 2% strain, MPa, @ 60° C. | 13.82 | 14.31 | 14.00 | 14.58 | 13.73 |
| Tan Delta at 2% strain | 0.174 | 0.174 | 0.170 | 0.170 | 0.167 |
| Shore A Hardness | 84 | 84 | 83 | 84 | 83 |
| Tensile Properties | | | | | |
| 100% Modulus, MPa | 4.02 | 4.55 | 4.43 | 4.62 | 4.49 |
| Tensile Strength, MPa | 26.2 | 27.7 | 27.1 | 27.6 | 27.7 |
| Elongation, % | 489 | 492 | 486 | 490 | 487 |
| Die C Tear, N/mm | 112 | 116 | 108 | 114 | 112 |

Note:
*B-20-S is PENACOLITE ® Resin B-20-S obtained from INDSPEC Chemical Corporation, Pittsburgh, PA.

### Example 8

### Synthesis of Alkylresorcinol-Styrene-Dual-Aldehyde Resins

TABLE 9
Synthesis of Alkylresorcinol-Styrene-Dual-Aldehyde Resins

Resin Number

| | |
|---|---|
| 15 | 16 |

| | |
|---|---|
| Raw Materials (Mole) | — |

| | | |
|---|---|---|
| HONEYOL | 1.37 | 1.37 |
| Styrene | 0.823 | 0.549 |
| Formaldehyde | 0.339 | 0.339 |
| Butyraldehyde | 0.663 | 0.663 |
| Resin Properties | | |
| Softening Point (° C.) | 95.6 | 86.3 |
| Free resorcinol (wt. %) | <0.02 | <0.02 |
| Free 2-methylresorcinol (wt. %) | <0.02 | <0.02 |
| Free 5-methylresorcinol (wt. %) | 0.5 | 0.1 |
| Free 2,5-dimethylresorcinol (wt. %) | 0.7 | 0.5 |

Alkylresorcinol-styrene-dual-aldehyde resins 15 and 16 were prepared according to the general procedures as described below. The molar charges of the ingredients for each resin are listed in Table 9 above.

First, the HONEYOL and p-toluene sulfonic acid at a level equal to 0.2 wt. % of HONEYOL were added to a reaction flask fitted with a stirrer, heating mantle and a condenser. The reflux from the condenser was set to return to the reaction flask. The HONEYOL was heated to 125° C. and stirred. Once the temperature of HONEYOL reached 125° C., styrene was added drop-wise to the flask over about 1 to 2 hours, while taking care to maintain the temperature between 125° C. and 135° C.

Once the styrene bad been added, the reaction mixture was briefly heated to 150° C. to assure the reaction was complete. The reaction mixture was cooled to about 115° C.

Butyraldehyde was then added to the reaction mixture over 1 to 1.5 hours, maintaining at about 110° C. to 115° C. The batch was then held at 115° C. for 30 minutes before cooling to about 100° C.

Next, formaldehyde was added drop-wise over 1 to 3 hours, so as not to exceed the capacity of the condenser. The reflux from the condenser was returned to the flask to cool the reaction material.

After all the formaldehyde was added, the p-toluene sulfonic acid was neutralized with an equal molar amount of sodium hydroxide solution. The condenser output was re-routed to a distillate receiver and the temperature was increased to distill water of reaction from the resin. Distillation continued at atmospheric pressure until the temperature of the reaction mass reached 140° C. to 145° C. Then, vacuum was applied to the flask to remove the remaining water. The batch was vacuum distilled to about 685 torr of vacuum and a temperature of 155° C. to 165° C., or until water content was below 2 wt. %.

After the vacuum was released, the resin was discharged and cast in a thin layer on a tray. After hardened, it was stored in a sealed jar. The resin was then tested for softening points and the amounts of free resorcinol and alkylresorcinols such as 2-methylresorcinol, 5-methylresorcinol and 2,5-dimethylresoreinol. After the testing, the resin was used in the rubber compounding experiments discussed below. The softening points and the amounts of free resorcinol, 2-methylresorcinol, 5-methylresorcinol and 2,5-dimethylresorcinol of alkylresorcinol-styrene-formaldehyde resins 15 and 16 are listed in Table 9 above.

### Example 9

### Testing of Alkylresorcinol-Styrene-Dual-Aldehyde Resins

Alkylresorcinol-styrene-formaldehyde resins 15 and 16 were used to prepare rubber compounds X and Y respectively according to the procedures described above, with the exception that the rubber compound used for this test did not contain the pre-vulcanization inhibitor shown as item 7 in Table 1, and the acceptor/donor ratios as shown in Table 10 below. Rubber compound W was also prepared similarly as a comparison using PENACOLITE® Resin B-20-S as the methylene acceptor. The physical properties of the rubber compounds were evaluated accordingly and the testing results are listed in Table 10 below. The data in Table 10 shows that the Mooney viscosity, dynamic mechanical properties at 2% strain, Shore A hardness values, tensile properties and Die C Tear properties of compounds W-Y are comparable. In rheometer cure, scorch safety of compounds X and Y are better than Compound W. The cure of compounds X and Y is slower than compound W. In wire adhesion, all un-aged samples are similar. In steam-aged adhesion, pullout force is equivalent but Compound X and Y are better in rubber coverage. In moisture-aged wire adhesion, pull-out force of all compounds are equivalent, but Compound X is lower than Compounds W and Y in rubber coverage.

TABLE 10

| | Compound | | |
|---|---|---|---|
| | W | X | Y |
| Methylene Acceptor | B-20-S* | Resin 15 | Resin 16 |
| Styrene/Alkylresorcinol mole ratio | 0.66:1 | 0.4:1 | 0.6:1 |
| Methylene Donor | HMMM | HMMM | HMMM |
| Weight Ratio; Acceptor/Donor, phr | 3/2 | 3/2 | 3/2 |
| Mooney Viscosity at 100° C. | | | |
| ML (1 + 4) | 64.9 | 62.9 | 62.3 |
| Rheometer Cure at 150° C. | | | |
| M_{H}, dN-m | 34.27 | 33.86 | 31.64 |
| M_{L}, dN-m | 3.17 | 3.01 | 2.85 |
| tₛ2, minutes | 2.26 | 2.71 | 3.32 |
| t′90, minutes | 18.21 | 20.30 | 22.27 |
| Wire Adhesion, N | | | |
| (% Rubber Coverage) | | | |
| Unaged | 1399(100) | 1407(95) | 1394(100) |
| Steam, 24 Hours @ 120° C. | 1012(50) | 1030(65) | 1056(70) |
| Humidity, 21 Days, 85° C./95% | 1006(65) | 1045(50) | 987(65) |
| RH | | | |
| Dynamic Mechanical Properties | | | |
| G′ at 2% strain, MPa, @ 23° C. | 17.88 | 18.13 | 17.46 |
| Tan Delta at 2% strain | 0.197 | 0.194 | 0.206 |
| G′ at 2% strain, MPa, @ 60° C. | 14.20 | 14.66 | 14.13 |
| Tan Delta at 2% strain | 0.187 | 0.189 | 0.200 |
| Shore A Hardness | 81 | 82 | 81 |
| Tensile Properties | | | |
| 100% Modulus, MPa | 4.82 | 4.94 | 4.75 |
| Tensile Strength, MPa | 25.5 | 26.1 | 25.1 |
| Elongation, % | 438 | 447 | 447 |
| Die C Tear, N/mm | 94 | 102 | 106 |

As demonstrated above, embodiments of the invention provide a modified alkylresorcinol resin for use in rubber compounding. The modified alkylresorcinol resin has lower softening points and therefore would enhance the processability of the uncured rubber compositions which incorporate the resin. However, the improved processability does not compromise other performance properties. For example, the adhesion properties, dynamic mechanical properties, tear properties of the uncured rubber composition are comparable or better than existing resorcinol-based resins. Accordingly, use of the modified alkylresorcinol resin in rubber compounding should yield better rubber products.

While the invention has been described with respect to a limited number of embodiments, the specific features of one embodiment should not be attributed to other embodiments of the invention. No single embodiment is representative of all aspects of the inventions. In some embodiments, the compositions may include numerous compounds not mentioned herein. In other embodiments, the compositions do not include, or are substantially free of, any compounds not enumerated herein. Variations and modifications from the described embodiments exist. The method of making the resins is described as comprising a number of acts or steps. These steps or acts may be practiced in any sequence or order unless otherwise indicated. Finally, any number disclosed herein should be construed to mean approximate, regardless of whether the word “about” or “approximately” is used in describing the number. The appended claims intend to cover all those modifications and variations as falling within the scope of the invention.

What is claimed is:

1. A modified alkylresorcinol resin prepared by a process comprising reacting a phenolic composition with (a) an olefinically unsaturated compound, and (b) at least an aldehyde, wherein the phenolic composition comprises from about 50 wt. % to about 100 wt. % of one or more alkylresorcinol compounds, from about 0 to about 20 wt. % of resorcinol, and from about 0 to about 10 wt. % of one or more monohydroxyphenol compounds as represented by formula (I) 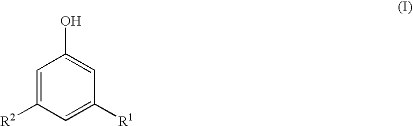
where each of R¹ and R²is independently H, alkyl, or OR³ where R³ is alkyl or aryl.

2. The modified alkylresorcinol resin of claim 1, wherein the alkylresorcinol compounds are represented by formula (II): 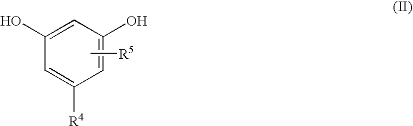
wherein R⁴ is alkyl or substituted alkyl; R⁵ is H, alkyl or substituted alkyl; and R⁵ is in 2, 4 or 6 position of the alkylresorcinol ring.

3. The modified alkylresorcinol resin of claim 2, wherein the phenolic composition comprises 5-methylresorcinol and 5-ethylresorcinol.

4. The modified alkylresorcinol resin of claim 1, wherein the phenolic composition comprises from about 1 wt. % to about 10 wt. % of the resorcinol.

5. The modified alkylresorcinol resin of claim 1, wherein the phenolic composition comprises from about 1 wt. % to about 9 wt. % of the monohydroxyphenol compounds.

6. The modified alkylresorcinol resin of claim 1, wherein the aldehyde is formaldehyde.

7. The modified alkylresorcinol resin of claim 1, wherein the aldehyde is a mixture of formaldehyde and an aldehyde represented by R⁷—CH═O, where R⁷ is a C₃₋₂₂ alkyl group.

8. The modified alkylresorcinol resin of claim 1, wherein the olefinically unsaturated compound is styrene.

9. The modified alkylresorcinol resin of claim 8, wherein the phenolic composition comprises 5-methylresorcinol and 5-ethylresorcinol and the aldehyde is formaldehyde.

10. A modified alkylresorcinol resin comprising a structure represented by one of the following formulae: 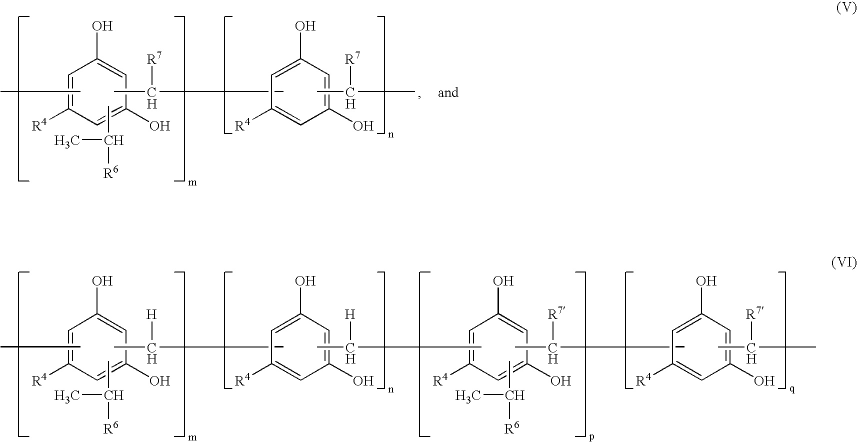
wherein R⁴ is alkyl; R⁶ is alkyl, substituted alkyl, aryl or substituted aryl; R⁷ is H, alkyl, substituted alkyl, aryl or substituted aryl; R^{7′} is alkyl or substituted alkyl; m and n are independently a positive integer; and p and q are independently zero or a positive integer, where the sum of m, n, p, and q is at least 3.

11. The modified alkylresorcinol resin of claim 10, wherein R⁶ is phenyl, R⁷ is H, and R^{7′} is propyl.

12. A vulcanizable rubber composition, comprising (a) a rubber component selected from natural rubber, synthetic rubber or a combination thereof, (b) a methylene donor compound, and (c) a methylene acceptor compound comprising a modified alkylresorcinol resin prepared by a process comprising reacting a phenolic composition with an olefinically unsaturated compound and at least an aldehyde, wherein the phenolic composition comprises from about 50 wt. % to about 100 wt. % of one or more alkylresorcinol compounds, from about 0 to about 20 wt. % of resorcinol, and from about 0 to about 10 wt. % of one or more monohydroxyphenol compounds as represented by formula (I) 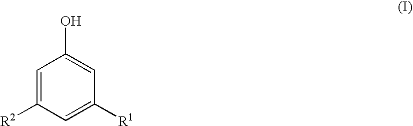
where each of R¹ and R² is independently H, alkyl, or OR³ where R³ is alkyl or aryl.

13. The vulcanizable rubber composition of claim 12, wherein the phenolic composition comprises 5-methylresorcinol and 5-ethylresorcinol.

14. The vulcanizable rubber composition of claim 12, wherein from about 30 mole % to about 65 mole % of the phenolic groups of the modified alkylresorcinol resin is aralkylated with the olefinically unsaturated compound.

15. The vulcanizable rubber composition of claim 14, wherein the olefinically unsaturated compound is styrene, α-methyl styrene, p-methyl styrene, α-chloro styrene, divinyl benzene, vinyl naphthalene, indene, vinyl toluene or a combination thereof.

16. The vulcanizable rubber composition of claim 12, wherein the methylene donor is hexamethylenetetramine, a methylol melamine, an etherified methylol melamine, an esterified methylol melamine, or a combination thereof.

17. The vulcanizable rubber composition of claim 12, wherein the phenolic composition comprises from about I wt. % to about 10 wt. % of the resorcinol.

18. The vulcanizable rubber composition of claim 12, wherein the phenolic composition comprises from about 1 wt. % to about 9 wt. % of the monohydroxyphenol compounds.

19. A vulcanizable rubber composition, comprising (I) a rubber component selected from natural rubber, synthetic rubber or combinations thereof (II) a methylene donor compound, and (III) a methylene acceptor compound comprising the modified alkylresorcinol resin of claim 10.

20. A process for making a modified alkylresorcinol resin, comprising reacting a phenolic composition with (a) an olefinically unsaturated compound, and (b) at least an aldehyde, wherein the phenolic composition comprises from about 50 wt. % to about 100 wt. % of one or more alkylresorcinol compounds, from about 0 to about 20 wt. % of resorcinol, and from about 0 to about 10 wt. % of one or more monohydroxyphenol compounds as represented by formula (I) 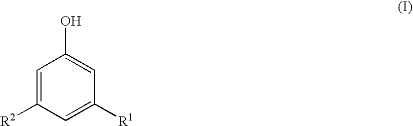
where each of R¹ and R² is independently H, alkyl, or OR³ where R³ is alkyl or aryl.

21. The process of claim 20, wherein the alkylresorcinol compounds are represented by formula (II): 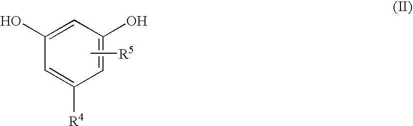
wherein R⁴ is alkyl or substituted alkyl; R⁵ is H, alkyl or substituted alkyl; and R⁵ is in 2, 4 or 6 position of the alkylresorcinol ring.

22. The process of claim 20, wherein the phenolic composition comprises 5-methylresorcinol and 5-ethylresorcinol.

23. The process of claim 20, wherein the aldehyde is formaldehyde.

24. The process of claim 20, wherein the aldehyde is a mixture of formaldehyde and an aldehyde represented by R⁷—CH═O, where R⁷ is a C₃₋₂₂ alkyl group.

25. The process of claim 20, wherein the olefinically unsaturated compound is styrene.
